(19) United States
(12) Patent Application Publication
(10) Pub. No.: US 2024/0077477 A1
(43) Pub. Date: Mar. 7, 2024

(54) Multi-Test Lateral Flow Assay Device

(71) Applicants: Terence Murphy, Doylestown, PA (US); Dilan Mehta, New York, NY (US)

(72) Inventors: Terence Murphy, Doylestown, PA (US); Dilan Mehta, New York, NY (US)

(21) Appl. No.: 18/238,457

(22) Filed: Aug. 25, 2023

Related U.S. Application Data
(60) Provisional application No. 63/400,946, filed on Aug. 25, 2022.

(51) Int. Cl. G01N 33/543

(52) U.S. Cl. CPC G01N 33/54388

(57) ABSTRACT

A lateral flow assay device that contains multiple test strips, allows multiple tests on a single fluid sample, and displays and transmits the test results to external monitors. The device receives a fluid sample and distributes it to multiple, independent test strips by capillary action. Each test strip has a conjugate pad, test line, and control line, that are designed to detect the presence of a specific analyte. Once the test is complete, photosensitive detectors convert the test results into electrical signals and transmit them to a motherboard. The motherboard then analyzes the test results to format appropriate messages, and transmits the messages to external monitors as well as to the device's own display screen.

### CROSS-REFERENCE TO RELATED APPLICATIONS

This nonprovisional patent application claims priority to U.S. provisional application 63/400,946, filed on Aug. 25, 2022, entitled Multiple Strip Linear Flow Assay with Central-Collar Analyte Pad for the Testing of Fluid Samples, the disclosure of which is considered part of the disclosure of this application and is hereby incorporated by reference in its entirety.

### FIELD OF THE INVENTION

This present invention relates to a lateral flow assay device for detecting multiple specific analytes of interest in a single fluid sample.

### BACKGROUND OF THE INVENTION

Testing for specific analytes in fluid samples is routinely performed on a commercial scale for various reasons, such as environmental monitoring, disease detection, and chemical analysis. It is also performed on a consumer level in, for example, healthcare products such as pregnancy tests and COVID-19 tests. Once known method, lateral flow assay, has emerged as a versatile method for a rapid and accurate detection of specific analytes in fluid samples. It has the potential to be adapted to diverse applications, including the detection of pollutants, chemicals, viruses, and other target analytes present in fluids.

Although there exists a need for a simple and comprehensive assay device that can reliably, accurately, and efficiently detect multiple analytes in a single fluid sample, such as when testing for multiple chemicals in a water sample, current state-of-the-art lateral flow assay devices are often physically limited to detecting only a single analyte per test sample. In addition, existing technologies for fluid testing often involve multiple handling steps, which may lead to spillage and contamination, and result in costly errors and wastes. Therefore, it is desirable to provide a lateral flow assay device that is easy to use and can detect multiple analytes of interest from a single test sample.

Many known lateral flow assays have a transparent window in which the results of the particular test are displayed. Those tests are often displayed visually as a single vs. double line, or blue vs. red colored field, etc. When those tests are performed on a commercial scale in high numbers, recording the test results by manually inputting them in a computer database can be costly and prone to human data entry error. Therefore, it is desirable to provide a lateral flow assay device that automatically communicates the test result to a computer database.

### SUMMARY OF THE INVENTION

The invention comprises a lateral flow assay device that can perform multiple tests on a single fluid sample admitted to the device. The device includes multiple test strips that are designed to detect the presence of a different specific analyte.

The multi-test lateral flow assay device includes a test housing and a nosepiece connected in fluid communication with the test housing. A plurality of lateral flow assay test strips are arranged within the test housing in isolation from one another. The device has means for dividing the fluid sample into sub-samples and conveying a sub-sample to each of the test strips. The device also has means for optically detecting the results displayed on each test strip and for automatically communicating those test results to an electronic data collection device.

In once preferred embodiment, the LFA test strips, distribution means and optical detection means are fixed within an elongate, cylindrical test housing. The nosepiece is removably fixed to the proximal end of the test housing. Communication means are located in a communications housing at the distal end of the test housing. The LFA test strips and other components that contact the fluid sample are removable and replaceable after each use, while all other components are re-usable since they do not contact the fluid sample.

In one preferred embodiment, the dividing and distribution means comprises a collar-shaped sample pad, which branches into multiple, integrally-formed test strips that have a backing to provide additional support. The collar-shaped sample pad accepts a fluid sample from the nosepiece and distributes a fluid sub-sample to the multiple test strips through capillary action. Each test strip preferably comprises an integrally-formed conjugate pad, test area, control area, and absorbent pad.

Preferably the optical detecting means comprises an array of photosensitive detectors positioned on the interior side of the test strips. The photosensitive detectors comprise a field of light emitting diodes and photodiodes, which read the test results by detecting, for example, the presence of a test line, or color change of a test line. Electric signals from the detectors are conveyed to a communications housing, which displays the results and/or conveys the results to an electronic data storage device.

In one typical assay session, a fluid sample enters the nosepiece and then flow to the sample pad. Through capillary action, the fluid sample flows from the sample pad to the proximal end of each test strip. The sample first binds with the conjugates in the conjugate pad on each test strip and continues to flow toward the test area. In the test area, the conjugated sample interacts with the reagents in the test line and changes the color of the test line if the test is positive. No change occurs to the test line if the test is negative. As the sample continues to flow further down the test strip, through capillary action and gravity, it interacts with the reagents in the control line and changes the color of the control line if the test is valid. No change occurs to the control line if the test is invalid.

At some point, the user turns on the electronic components of the device, powering up a motherboard, the photosensitive detectors, a communication unit, and a display screen. The photosensitive detectors measure the color of the test line and send an electronic signal to the motherboard, which then interprets the signal and displays a corresponding message on the display screen. The communication unit also transmits a message to a nearby monitor paired with the device for storage, analysis, and additional display.

### BRIEF DESCRIPTION OF THE DRAWINGS

FIG. 1 is a perspective view of a multi-test lateral flow assay device in accordance with one preferred embodiment of the invention;

FIG. 2 is an exploded perspective view of the multi-test lateral flow assay device of FIG. 1;

FIG. 3 shows various perspective views of the test strips of the multi-test lateral flow assay device of FIG. 1;

FIG. 4 is a cross-sectional view taken along lines 4-4 of FIG. 1;

FIG. 5 is a flowchart illustrating the testing method in accordance with an embodiment of the invention;

FIG. 6a is a perspective view of a multi-test lateral flow assay device in accordance with another preferred embodiment of the invention;

FIG. 6b is an exploded wireframe view of the device shown in FIG. 6a;

FIG. 7 is an exploded perspective view of the multi-test lateral flow assay device in accordance with another preferred embodiment of the invention; and

FIG. 8 is a cross-sectional schematic view of an alternative arrangement of the photosensitive detectors in the test area of a device in accordance with another embodiment of the invention.

### DESCRIPTION OF PREFERRED EMBODIMENTS OF THE INVENTION

For the purpose of illustrating the invention, several embodiments of the invention are described with respect to the accompanying drawings. However, it should be understood by those of ordinary skill in the art that the invention is not limited to the precise arrangements and instrumentalities shown therein and described below. Throughout the specification, like reference numerals are used to designate like elements.

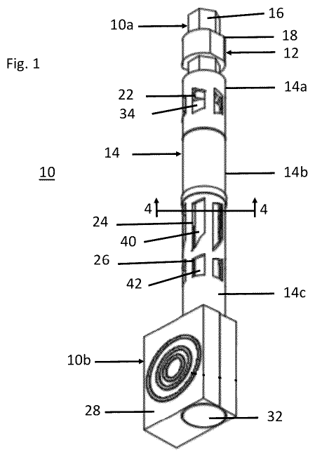

A multi-test lateral flow assay device in accordance with a preferred embodiment of the invention is shown in FIGS. 1-8 and is designated generally by reference numeral 10. While an embodiment of the device 10 having six separate lateral flow assay (“LFA”) test strips is illustrated and described in FIGS. 1-8, it should be appreciated by those of ordinary skill in the art that the device 10 may have more than or less than six LFA test strips without departing from the scope of the invention.

With reference to the orientation shown in FIG. 1, the device has an upper, proximal end 10a in which a fluid sample is admitted, and a lower distal end 10b from which excess fluid sample is emitted. The multi-test LFA device generally comprises a fluid-sample collection nosepiece 12, a plurality of LFA test strips, means for dividing the fluid sample and distributing a portion of the fluid sample to each of the LFA test strips, means for optically detecting the result on each test strip, and means for automatically communicating those test results to a digital data collection device. In the preferred embodiment shown in FIG. 1, the LFA test strips, distribution means and optical detection means are fixed within an elongate, cylindrical test housing 14. The nosepiece is removably fixed to the proximal end of the test housing 14. The communication means are located in a communications housing 28 at the distal end of the test housing 14. The LFA test strips and other components that contact the fluid sample are removable and replaceable after each use, while all other components are re-usable since they do not contact the fluid sample.

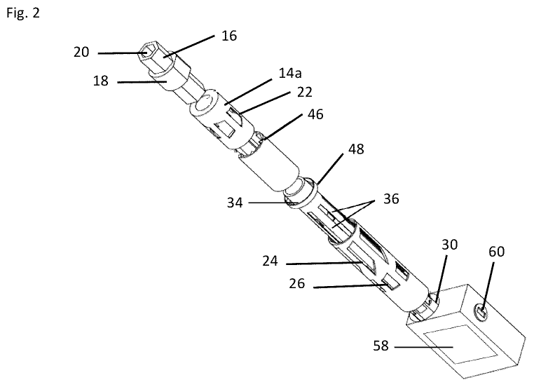

In the embodiment shown in FIGS. 1 and 2, the nosepiece 12 comprises an elongate, sample collection tube 16 and a lip rest 18 surrounding the outer surface of the collection tube 16. The nosepiece is designed to collect a fluid sample in a variety of ways. For example, a patient can insert the collection tube 16 in his/her mouth and expelling saliva into the proximal port 20 of the collection tube 16. The lip rest 18 prevents the patient from inserting the nosepiece too far into the patient's mouth. Alternatively, a fluid sample from a dispenser, such as a pipettor, can be deposited into the nosepiece 12. Other methods of collecting a fluid sample could include insertion of a buffered absorbent swab into the nosepiece 12, or aspirating a salivary sample from a patient by connecting a dental saliva ejector mouthpiece to the nosepiece 12 and connecting a suction source to the discharge port 32 (discussed below) at the other end. Regardless of the method of collecting the fluid sample, in preferred methods the device is held generally vertically as shown in FIG. 1 so that the fluid sample flows downwardly and evenly through the nosepiece and into the test housing 14.

The nosepiece 12 inserts into and removably connects in fluid communication with the proximal end of the test housing 14. The test housing 14 can be made as a single component but is preferably made from interconnecting subcomponents 14a-c, as best seen in FIG. 1, which makes replacement of the internal test strips/assays easier. In a preferred embodiment shown in FIGS. 1-8, the test housing 14 comprises first 14a, second 14b, and third 14c housing components that interconnect using known techniques. Each of the housing components comprises a generally cylindrical, thin-walled tube made of rigid material such as plastic. To avoid cross-contamination of the test strips, the housing material should preferably be a hydrophobic material.

The first 14a housing component has a plurality of radially-spaced apertures 22, which function as secondary fluid-sample collection ports. For example, if the nosepiece 12 is inserted into a patient's mouth past the lip rest and far enough that the apertures 22 enter the patient's mouth, excess saliva can flow through the apertures 22 as well as through the distal port 20 of the sample collection tube 16. As described below, the first distribution stage of the multi-test LFA test device is located within the first housing component 14a. Therefore, the collection tube 16 and ports 22 are constructed and arranged to collect and channel the fluid sample to that first test stage.

The third housing component 14c also has a plurality of radially-spaced apertures 24, 26, which function as observation windows. As described below, the third, test stage of the multi-test LFA test device is located within the third housing component 14c. The windows allow the user to visually observe the test reading/result that appears on the internal test strips. However, in order to avoid contamination from external sources, the observation apertures 24, 26 have transparent covers such as shown in FIGS. 1-2. In an alternative embodiment, the third component may be transparent to eliminate the need to form and cover the observation apertures 24, 26.

The third housing component 14c is connected to the communications housing 28. In one preferred embodiment, the third housing component 14c inserts into a fluid-flow channel 30, which extends through the communications housing 28 and terminates at the distal discharge port 32. As described in greater detail below, the communications housing 28 contains known electronic and computing equipment to display the test results on a display screen 58 as well as transmitting those results to an external digital storage device.

A multi-test lateral flow assay device is located within the test housing 14. In the embodiment shown in FIGS. 1-8, the assay device is constructed to perform six separate reagent tests on the single fluid sample using known LFA techniques. However, it should be appreciated that the device could be constructed and arranged to perform more than or less than six tests using the same structural concepts described and illustrated herein.

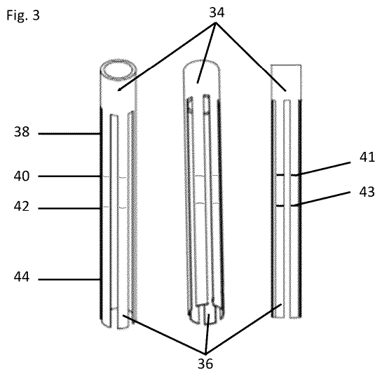

In the first stage of the multi-test LFA, the single fluid sample is divided into a predetermined number of smaller samples (subsamples), and each subsample is distributed to individual test strips. In one preferred embodiment, the means for dividing and distributing the fluid subsamples comprises a sample pad 34 having a collar-shape or cylindrical-shape as best seen in FIG. 3. The sample pad 34 is positioned in the first test housing portion 14a proximate to the distal end of the fluid collection tube as see in FIG. 1. As fluid exits the collection tube into the first housing portion 14a, or enters the first housing portion through the apertures 22, it contacts and is absorbed by the sample pad 34. The fluid sample is then divided and evenly distributed around the periphery and axial length of the sample pad 34.

In the second stage, the subsamples are conveyed to each of the six individual test strips 36. In this preferred embodiment, the test strips 36 are integrally formed and branch axially outwardly from the sample pad 34 as best seen in FIG. 3. The sample pad 34, as well as each test strip 36, is made of nitrocellulose membrane or other material such as rayon depending on the required flow characteristics of the fluid sample, hydrophilicity, biodegradation, and cost, which allows a fluid sample to flow from one end to the other end of the test strips 36. Therefore, in this embodiment, transmission from the sample pad 34 to the test strips 36 is achieved by capillary action, which is assisted by gravity when the device 10 is held upright in the orientation shown in FIG. 1.

In the third stage, the subsamples react with reagents using known LFA technology to test for a variety of conditions. As best seen in FIG. 3, each test strip 36 consists of a conjugate pad 38, test area 40 containing a test line 41, control area 42 containing a control line 43, and an absorbent pad 44. In other preferred embodiments, the test strips 36 have two or more test lines 41 depending on the type of analytes and testing requirements. As best seen in FIG. 1, the conjugate pad is located within the second test housing portion 14b. The test area 40 and control area 42 are located within the third test housing portion 14c, and are positioned so that the test line 41 and control line 43 portions are aligned within one of the observation windows, 24, 26, respectively. By allowing the test line 41 and control line 43 to be viewable through the windows 24, 26, the user can visually confirm the test results reported electronically, discussed below. The absorbent pad 44 is also preferably located within the distal, solid portion of the third test housing portion 14c.

In addition to the test strips 36, the multi-test lateral flow assay device may have adulterant detection strips, which are similar in size and shape to the test strips 36 but have adulterant detection means. Such an adulterant detection strip can determine whether a sample has been tampered with, or contaminated, which may interfere with test results. Examples of adulterant detection means include: pH indicator strips, specific chemical reagents, temperature sensors, specific biomarkers, dilution detectors, specific enzyme activity sensors, conductivity sensors, and immunoassays.

The sample pad 34 and test strips 36 are stabilized within the test housing 14 by axially-extending, stabilizing tongs 46 and test strip stabilizing cap 48. The tongs 46 form pockets into which each test strip 36 is seated and separated from an adjacent strip to prevent cross-contamination. In place of the stabilizing tongs 46 and stabilizing cap 48, other stabilizing means can be used such as clips, latches, hooks, enclosures, snaps, or an adhesive, along the inner walls of the test housing 14.

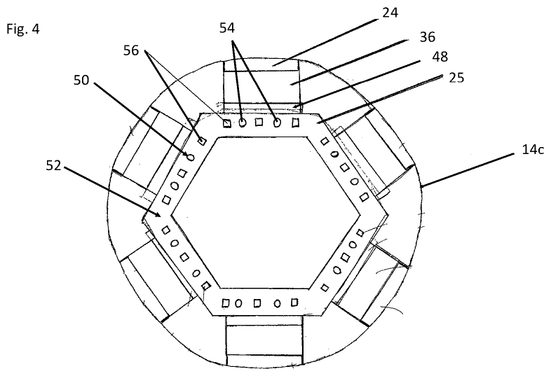

As shown in FIG. 4, a backing 48 is attached to the inside of each test strip 36 by an adhesive or other means to provide rigidity to the test strips 36. The backing 48 is made from a transparent material so as not to interfere with the photosensitive detectors described below.

With further reference to FIG. 4, an array of photosensitive detectors 50 is mounted on each side of a hexagonal frame 52 positioned on the interior side of the test strips 36. The photosensitive detectors 50 comprise a field of light emitting diodes (LEDs) 54 and photodiodes 56. The array of detectors 50 extends both radially and axially in the area adjacent the test line 41 and control line 43 so that the detectors 50 can read the test results by detecting, for example, the change of colors of the test line 41 and control line 43.

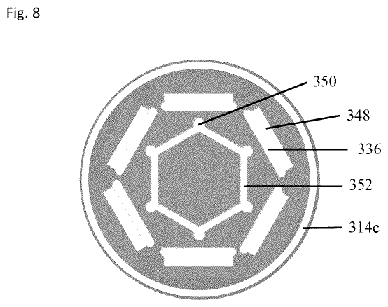

In another embodiment shown in FIG. 8, the number of detectors is reduced by replacing each field of detectors 50 with a linear array 350 of detectors on the inner frame 352. In this alternative embodiment, each array consists of a pair of photosensitive detectors 350, one LED and one photodiode, axially positioned directly in front of the test line 41 and in front of the control line 43. In this embodiment shown in FIG. 8, the detectors 350 are positioned along the corners of the frame, but alternatively may be positioned along the flat surfaces of the frame 352.

The photosensitive detectors 50 are electrically wired to a known motherboard and communication unit within the communications housing 28 to analyze the photodetection signals and record the test results. The communications housing 28 preferably also includes a display screen 58 for displaying the results and other device-related messages. In addition, or alternatively, the test results are transmitted to an external data collection device such as a computer, phone, or other devices for the storage, analysis, and display of the test results. The communication unit may use near field communication (NFC), Bluetooth, Wi-Fi, or other wireless communication protocols.

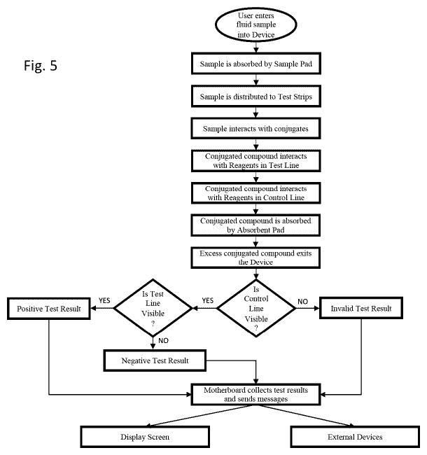

A testing process in accordance with an embodiment of the multi-test lateral flow assay device 10 is illustrated in FIG. 5. First, the user collects a fluid sample, which flows to and is immediately absorbed by the sample pad 34. While keeping the device 10 upright throughout the testing process, the sample pad 34 may be treated to adjust the sample's properties such as the pH level or viscosity. Through capillary action, and to a lesser extent by gravity, the fluid sample is divided by and dispersed from the sample pad 34 to each of the six test strips 36. the fluid sub-samples then flow to the conjugate pads 38, which contain particles that are coated with reagents tailored to specific analytes of interest. The reagents contain labels made of colloidal gold, carbon, or latex, depending on the compatibility with the analytes of interest. Since each test strip 36 is separated from each other, the risk of cross-conjugate contamination is minimized. As the sample passes through the conjugate pad 38, the conjugate particles bind with the target analyte, forming a conjugate compound. Other known examples of reagents include aptamers, enzymes, molecularly imprinted polymers, fluorescent dyes, and synthetic peptides.

The conjugate compound then enters the test area 40 where the conjugate compound interacts with the capture reagents contained in the test line 41. If no binding occurs between the conjugate compound and the capture reagents at the test line 41 then the test result is negative. If the conjugate compound and the capture reagents at the test line 41 bind then the labels give color to the test line 41, indicating a positive result.

The conjugate compound then enters the control area 42 where the conjugate compound interacts with the capture reagents contained in the control line 43. If no binding occurs between the conjugate compound and the capture reagents at the control line 43, then the test result is invalid. If the conjugate compound and the capture reagents at the control line 43 bind then the labels give color to the control line 43, indicating that the test has run correctly.

The conjugate compound leaves the control area 42 and reaches the absorbent pad 44. The absorbent pad 44 helps with the flow of the fluid sample and collects any excess sample. Any excess sample that is not entirely collected by the absorbent pad 44 exits through the discharge port 32 where the sample may be discarded, or re-collected for additional testing and analysis.

Sometime prior to, or soon after, the completion of the assay test, the user powers up the electronic components of the device 10 by switching on the power switch 60. Then, the photosensitive detectors 50 measure the light wavelengths of the test line 41 and control line 43 and convert the measurements to electrical signal. More specifically, the LEDs 54 emit light onto the test line 41 and control line 43 and the photodiode sensors 56 measure the wavelength of the light from those lines, and then convert the measurement to electrical signals, which are interpreted by the motherboard and displayed as test results on the display screen 58 indicating a positive result, a negative result, or an invalid result, for each of the six test strips 36. Optionally, if one or more external devices have been paired with the device, then the motherboard also sends the test data to such external devices via the communication unit.

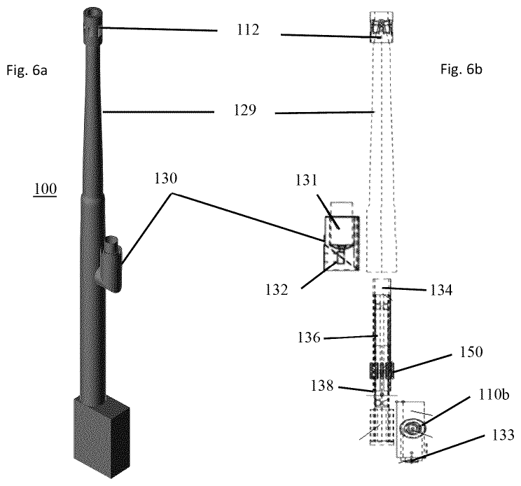

FIGS. 6a and 6b show a multi-test linear flow assay device 100 in accordance with another embodiment of the invention. This embodiment of the device 100 is structurally and functionally similar to the device 10 described above with respect to FIGS. 1-5. However, in this embodiment, the device 100 has an extended fluid collection nosepiece 112 and a nebulizer 130. The nosepiece 112 is hollow and facilitates the flow of a fluid sample from the source to the sample pad 134. The extended nosepiece 112 helps easily collect test sample fluid from tight spaces such as a human mouth. If necessary, a suction hose such as one used in a dental office may be attached to the discharge port 32 to provide a negative pressure to the nosepiece 112 and help facilitate the collection of a fluid sample.

The nebulizer 130 has a nebulizer cartridge port 132, which serves as an entry point into which a nebulizer cartridge 131 is attached. The nebulizer cartridge port 132 includes a securing means such as clips, latches, or a locking mechanism that securely engages with the nebulizer cartridge 131 and prevents accidental dislodging or detachment of the nebulizer cartridge 131 during operation or storage.

During the fluid collection step, the user activates the nebulizer 130 by pressing the nebulizer cartridge 131 into the nebulizer cartridge port 132. The nebulizer 130 then converts the fluid sample into mist so that the sample is more easily absorbed by the sample pad 134 and the test strips 136 and to better facilitate interactions between the sample and the reagents. The nebulizer cartridge 131 may contain compressed air or other materials specific to the analytes of interest.

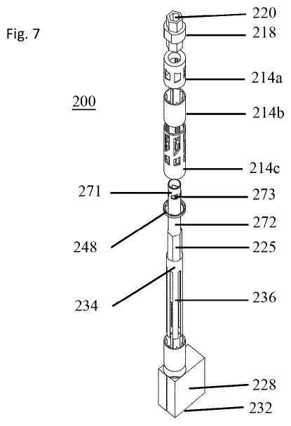

An additional preferred embodiment of the multi-test linear flow assay device 200 is shown in FIG. 10. This embodiment of the device 200 is structurally and functionally similar to the device 10 described above with respect to FIGS. 1-5. Elements in this embodiment that are identical to an element in the device 10 of FIGS. 1-5 are identified using the same reference numeral preceded by the numeral “2”. For example, the identical nosepiece 18 of the device 10 is identified using reference numeral 218 in the embodiment 200 shown in FIG. 7.

In this embodiment, the device 200 includes a flow controller 271, which is used to divide the incoming fluid sample when the fluid sample is expected to be large in volume, such as when collecting saliva in a dental office using a suction hose. The flow controller 271 is located inside the first housing component 214a and on the inside of the sample pad 234. Its proximal end is attached to the distal end of the nosepiece 218 such that any fluid entering through the proximal port of the collection tube 220 also enters the flow controller 271. The flow controller 271 extends into the flow pipe 272, which extends to the discharge port 232 so that any fluid traveling from the flow controller 271 down the flow pipe 272 can reach the discharge port 232 without contacting any other part of the multi-test lateral flow assay device 200.

The flow controller 271 has small windows 273 that divert the flow of the fluid sample into two streams. One stream flows out the windows and immediately reaches the sample pad 234. The other stream flows down the flow pipe 272 and exits the discharge port 232.

Having described the preferred aspects and implementations of the present disclosure, modifications and equivalents of the disclosed concepts may readily occur to one skilled in the art. However, it is intended that such modifications and equivalents be included within the scope of the claims which are appended hereto.

1. A multi-test lateral flow assay device for performing multiple tests on a single fluid sample, comprising:
a. a test housing;
b. a fluid-sample collection nosepiece connected in fluid communication with said test housing;
c. a plurality of isolated lateral flow assay test strips within said test housing;
d. means for dividing the fluid sample into sub-samples and conveying a sub-sample to each of said test strips;
e. means for optically detecting the results displayed on each test strip; and,
f. means for automatically communicating those test results to an electronic data collection device.
